(19) United States
(12) Plant Patent Application Publication
(10) Pub. No.: US 2016/0227690 P1
(43) Pub. Date: Aug. 4, 2016

(54) Origanum Plant Named 'Bellissimo'

(50) ;

(71) Applicant: Marcus Harvey, South Hobart (AU)

(72) Inventor: Marcus Harvey, South Hobart (AU)

(21) Appl. No.: 14/544,685

(22) Filed: Feb. 3, 2015

(51) Int. Cl. A01H 5/00

(52) U.S. Cl. PLT/258

(57) ABSTRACT

A new and distinct Origanum cultivar named ‘BELLISSIMO’ is disclosed, characterized by a controlled, attractive, dome shaped growth habit. Flowers have large dark pink floral bracts. The new cultivar is an Origanum, typically suited for garden and ornamental container use.

### LATIN NAME OF THE GENUS AND SPECIES

Origanum hybrid

### VARIETY DENOMINATION

‘BELLISSIMO’

### BACKGROUND OF THE INVENTION

The new cultivar is a product of a planned breeding program. The new variety originated from the pollination of the seed parent, an unpatented variety of Origanum hybrid known as ‘Kent Beauty’ with the pollen parent, an unpatented variety of Origanum hybrid known as ‘Barbara Tingey’ conducted by the inventor during 2009. The crossing was made in a commercial nursery in South Hobart, Tasmania, Australia.

‘BELLISSIMO’ was first discovered by the inventor, Marcus Harvey, in 2010 at the same nursery in South Hobart, Australia. The inventor observed the new variety for three years, making the final selection of the variety during 2013.

Asexual reproduction of the new cultivar ‘BELLISSIMO’ was first performed in the nursery at South Hobart, Australia by vegetative tip cuttings during 2010. ‘BELLISSIMO’ has since produced at least six generations and has shown that the unique features of this cultivar are stable and reproduced true to type.

### SUMMARY OF THE INVENTION

The cultivar ‘BELLISSIMO’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Bellissimo’. These characteristics in combination distinguish ‘BELLISSIMO’ as a new and distinct Origanum cultivar:

1. Controlled, attractive dome shaped plant habit.
2. Deep pink bract coloration

### PARENTAL COMPARISON

Plants of the new cultivar ‘BELLISSIMO’ are similar to the female parent ‘Kent Beauty’ most horticultural characteristics. However, ‘BELLISSIMO’ differs in producing bracts of a much deeper pink color than the seed parent.

Plants of the new cultivar ‘BELLISSIMO’ are similar to the male parent ‘Barbara Tingey’ in most horticultural characteristics. However, plants of ‘BELLISSIMO’ are dome shaped in habit, whereas plants of the pollen parent are prostrate to trailing.

### COMMERCIAL COMPARISON

‘BELLISSIMO’ is best compared to the parent varieties, which are well known.

### BRIEF DESCRIPTION OF THE PHOTOGRAPHS

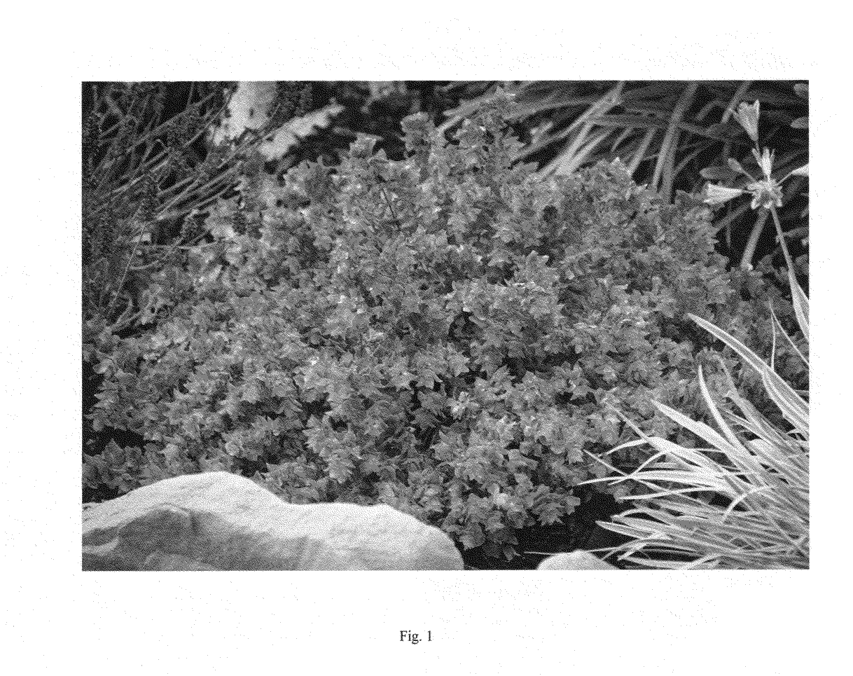

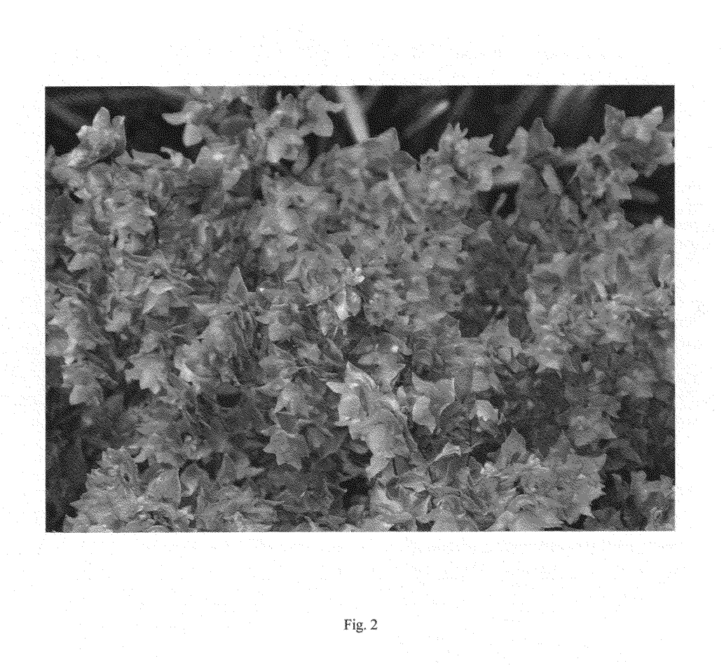

The accompanying photograph in FIG. 1 illustrates in full color a typical plant of ‘BELLISSIMO’ grown outdoors in South Hobart, Australia. This plant is approximately 1 year old, shown growing in the ground. FIG. 2 illustrates a close up view of the floral bracts. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘BELLISSIMO’ plants grown in a outdoors in South Hobart, Australia. Temperatures ranged from 5° C. to 18° C. at night and approximately 10° C. to 30° C. during the day. No artificial light, photoperiodic treatments or chemical treatments were given to the plants. Measurements and numerical values represent averages of typical plant types.
Botanical classification: Origanum hybrid ‘Bellissimo’
   Age of the plant described.—Approximately 1 year.
   Container size of the plant described.—approximately 2 liter pot.
Propagation:
   Time to initiate roots.—About 5 days at temperatures of 20 to 25 degrees C.
   Time to develop roots.—About 20 days at temperatures of 20 to 25 degrees C.
   Root color.—Youngest roots and root hairs near RHS White 155A mature roots near Brown 200B.
   Root description.—Very thin and fibrous.
Plant:
   Growth habit.—Fleshy annual or tender perennial, forming an even dome shape.
   Height.—Measured from top soil line of pot, approximately 28 cm to top of highest leaf. Approximately 35 cm to top of highest inflorescence.
   Plant spread.—Approximately 45 cm.
   Growth rate.—Rapid.
   Branching characteristics.—Very well branched. Approximately 20 to 30 branches.
   Length of primary branches.—About 30 cm.
   Diameter of primary branches.—About 0.3 cm.
   Characteristics of primary lateral branches.—Form: Cylindrical Diameter: About 0.3 cm. Color: Near RHS Green 143A Texture: Pubescent Strength: Strong, flexible.
   Internode length.—Average range about 1.3 to 2.0 cm.
Foliage:
   Leaf.—Arrangement: Simple, opposite Average Length: Approximately 1.9 cm. Average Width: Approximately 1.4 cm. Shape of blade: Ovate to somewhat deltate. Apex: Acute. Base: Blunt. Margin: Entire. Texture of top surface: Pubescent, matte. Texture of bottom surface: Pubescent, matte. Quantity of leaves per branch: Approximately 15. Color: Young foliage upper side: Near RHS Green 137A. Young foliage under side: Near RHS Green 137A. Mature foliage upper side: Near RHS Green 137A. Mature foliage under side: Near RHS Green 137A.
   Venation.—Type: Pinnate. Venation color upper side: Near RHS Yellow-Green 144A. Venation color under side: Near RHS Yellow-Green 144A.
   Petiole.—Not present, sessile.
Flower:
   Natural flowering season.—Spring and Summer.
   Inflorescence.—Form: Axillary, compact cyme, large, ornamental bracts. Width of entire inflorescence: Approximately 5 cm Height of entire inflorescence: Approximately 9 cm Quantity of individual flowers in inflorescence: Average range 10 to 15.
   Quantity of inflorescence per plant.—Average range 20 to 50.
   Persistent or self-cleaning.—True flowers self-cleaning, bracts persistent.
   Peduncle.—Length: Approximately 3.5 cm. Width: Approximately 0.3 cm. Color: Near RHS Yellow-Green 144B. Texture: Pubescent Strength: Flexible.
   Flowers.—
   Bud.—Shape: Deltate Length: 0.6 cm Diameter: 0.3 cm Color: Near RHS Red-Purple 61A.
   Bracts.—Quantity: Average 2. Arrangement: Opposite. Length: Approximately 1.6 cm Width: Approximately 0.9 cm. Shape: Deltate Apex: Acute. Base: Oblique. Margin: Entire. Texture: Glabrous. Appearance: Slightly shiny. Color: Inner Surface: Near Red-Purple 60D Outer surface: Near Red-Purple 60C.
Reproductive organs: Not observed.
Other characteristics:
   Seeds.—Seed production not observed.
   Disease/pest resistance.—Neither resistance nor susceptibility to common diseases and pets of Origanum have been observed in the new variety.
   Temperature tolerance.—Tolerates temperatures from approximately 4° C. to 38° C.

What is claimed is:

1. A new and distinct cultivar of Origanum hybrid plant named ‘BELLISSIMO’ as herein illustrated and described.
